(19) United States
(12) Patent Application Publication
(10) Pub. No.: US 2009/0234368 A1
(43) Pub. Date: Sep. 17, 2009

(54) LOW PROFILE MEDICAL DEVICES WITH INTERNAL DRIVE SHAFTS THAT COOPERATE WITH RELEASABLY ENGAGEABLE DRIVE TOOLS AND RELATED METHODS

(76) Inventor: Brian Gore, Fort Lauderdale, FL (US)

Correspondence Address: Boston Scientific Neuromodulation (SVI & JHU);c/o Darby & Darby P.C., P.O. Box 770, Church Street Station, New York, NY, 10008-0770, US

(21) Appl. No.: 12/403,744

(22) Filed: Mar. 13, 2009

Related U.S. Application Data
(60) Provisional application No. 61/037,084, filed on Mar. 17, 2008.

(51) Int. Cl. A61N 1/05; A61N 1/02

(52) U.S. Cl. 606/129; 607/127

(57) ABSTRACT

The disclosure describes medical tools such as implantable leads that have internal drive shafts for deploying an extendable member and associated clinician tools for engaging the drive shaft.

### RELATED APPLICATION

This application claims the benefit of priority to U.S. Provisional Application Ser. No. 61/037,084, filed Mar. 17, 2008, the contents of which are hereby incorporated by reference as if recited in full herein.

### BACKGROUND

Medical leads can have active fixation ends that extend to engage local tissue during a surgical procedure such as placement of implantable leads in the body for cardiac pace-making. In the past, an inner conductor has been configured to rotate to extend the screw end out of the lead while applying torque. There remains a need for alternate designs that allow for low profile lead configurations.

### SUMMARY OF EMBODIMENTS OF THE INVENTION

Embodiments of the present invention are directed to medical leads with integral drive shafts that can rotate. The leads can be low-profile and flexible and may be MRI-safe.

Some embodiments are directed to medical devices that include: (a) an elongate body having opposing proximal and distal end portions with an axially extending center cavity; (b) an internal drive shaft residing in the center cavity, the drive shaft having a proximal end portion with a rotatable spline or spline engagement member residing in the proximal end portion of the lead; and (c) an extendable member held in a retracted configuration in the distal end portion of the body, the extendable member in communication with the drive shaft whereby rotation of the drive shaft causes the extendable member to advance to extend out of the body.

In some embodiments, the elongate body can be an intrabody medial lead that can have low DC resistance and can be flexible. The extendable member can include or be a fixation screw, such as, for example, an active fixation screw electrode.

In some embodiments, the extendable member is an electrode or sensor and the medical device can include at least one conductor in electrical communication with the electrode or sensor. The elongate body can be implantable and have a diameter that is less than about 0.10 inches over at least a major portion of its length.

The elongate body can be an implantable neuromodulation lead or an implantable cardiac lead. In particular embodiments, the lead is an implantable pacemaker lead.

In some embodiments, the lead can include a flexible inner sleeve residing over the drive shaft and at least one coiled conductor portion coiled substantially concentrically about the sleeve.

In some embodiments, the lead can include a stationary electrode disposed on the proximal end portion of the lead about the drive shaft spline or spline engagement member.

In some embodiments, the elongate body can be used in combination with a single-use disposable drive tool, the drive tool having a primary tool body with an axially extending cavity and an integral spline or spline engagement member residing in a distal end portion of the tool body. The tool spline or spline engagement member can be adapted to slidably releasably engage the lead spline or spline engagement member whereby a user can rotate the drive shaft.

The tool body may include a bore sized and configured to snugly slidably receive the proximal end portion of the lead. The bore can have a larger diameter than the axially extending cavity. The tool body can receive a stylet that extends out of the proximal end portion of the tool and is connected to the drive shaft.

Other embodiments are directed to surgical tool sets. The tool sets include: (a) a flexible (intrabody) medical lead having an internal drive shaft with a spline or spline engagement member, the lead also comprising a plurality of electrodes, and a plurality of conductors, each electrode in communication with at least one of the conductors; and (b) a drive tool having an internal spline or spline engagement member sized and configured to slidably releasably engage the drive shaft spline or spline engagement member. The drive tool can include a cavity for receiving a stylet that is configured to allow a user to translate the drive shaft of the medical lead.

The tool set may include a second drive tool held in a discrete sterile package for future use. The lead may include an active fixation device configured to attach to local tissue on a distal end thereof in communication with the drive shaft, wherein the drive shaft rotates causing the active fixation device to extend or retract relative to the distal end or the lead.

Still other embodiments are directed to methods of advancing an extendable member from a medical lead. The methods include: (a) matably engaging an integral spline of a disposable single-use drive tool with an intrabody medical lead having an internal drive shaft and spline; (b) turning the drive tool to rotate the drive shaft; and (c) rotating the extendable member from the lead in response to the turning step.

The methods may also include turning the tool in a direction opposite of that used to advance the extendable member and retracting the extendable member back into the lead in response thereto.

Yet other embodiments are directed to an implantable pacemaker lead that includes: (a) a medical lead having opposing proximal and distal end portions with an axially extending center cavity; (b) an internal drive shaft residing in the center cavity, the drive shaft having a proximal end portion with a rotatable spline residing in the proximal end portion of the lead; and (c) an extendable member held in a retracted configuration in the distal end portion of the lead, the extendable member in communication with the drive shaft whereby rotation of the drive shaft causes the extendable member to translate.

The lead may have low DC (direct current) resistance and may be flexible. The extendable member can include a screw electrode and the lead can have a diameter that is less than about 0.10 inches over at least a major portion of its length.

Still other embodiments are directed to a single-use disposable medical drive tool having an internal spline or spline engagement member sized and configured to slidably releasably receive and engage an end portion of a medical lead having a drive shaft with spline or spline engagement portion. The drive tool further comprises a cavity for receiving a stylet. The drive tool is configured to allow a user to rotate the drive shaft of the medical lead. The medical drive tool is held in a sterile package.

Further features, advantages and details of the present invention will be appreciated by those of ordinary skill in the art from a reading of the figures and the detailed description of the embodiments that follow, such description being merely illustrative of the present invention.

### DRAWINGS

FIG. 1 is a partial cutaway, partial transparent side view of a lead having a driveshaft according to embodiments of the present invention.

FIG. 2 is a partially transparent end perspective view of the lead shown in FIG. 1.

FIG. 3 is a sectional side view of the proximal end of the lead shown in FIG. 1.

FIG. 4A is a sectional side view of a drive tool according to embodiments of the present invention, illustrating the lead shown in FIG. 1 aligned but not fully engaged according to embodiments of the present invention.

FIG. 4B is a sectional side view of the device shown in FIG. 4A, illustrating the lead shown in FIG. 1 in operative position according to embodiments of the present invention.

FIG. 5A is partial transparent and cutaway side view of the proximal end of the lead shown in FIG. 1 with the drive shaft in a retracted configuration according to embodiments of the present invention.

FIG. 5B is a partial transparent and cutaway side view of the device shown in FIG. 5A with the drive shaft in an extended configuration according to embodiments of the present invention.

FIG. 6 is a partial side sectional view of a portion of the lead shown in FIG. 1, including the proximal portion.

FIG. 7 is a partial side sectional and partially transparent view of the portion of the lead shown in FIG. 6 illustrating additional features according to embodiments of the present invention.

FIG. 8 is a partially transparent side view of the lead shown in FIG. 1 with the distal portion shown according to embodiments of the present invention.

### DETAILED DESCRIPTION

The present invention now is described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity. Broken lines illustrate optional features or operations unless specified otherwise.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and should not be interpreted in an idealized or overly formal sense unless expressly so defined herein. Well-known functions or constructions may not be described in detail for brevity and/or clarity.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention. The sequence of operations (or steps) is not limited to the order presented in the claims or figures unless specifically indicated otherwise. Certain of the figures illustrate the device as partially transparent (the affected components so shown indicated by broken lines) for ease of reference to internal components.

The term “drive shaft” refers to a rotating member that transmits torque or otherwise advances and/or retracts a target member. The term “spline” refers to a series of projections on a shaft that fit into slots or mating projections on a corresponding shaft, thereby allowing both to rotate together while one shaft translates relative to the other. Thus, one shaft can have a first spline and a second shaft can have a matably engaging spline or a spline engagement member. The spline or spline engagement member can comprise slots, projections and/or recesses and the like, that engage the target spline to allow both shafts to rotate together while translating relative to each other.

The term “lead” refers to an elongate assembly that includes one or more conductors. The lead typically connects two spaced apart components, such as, for example, a power source and/or input at one end portion and an electrode and/or sensor at another position, such as at a distal end portion, or electrodes at both end portions. The lead is typically flexible. The lead can be substantially tubular with a cylindrical shape, although other shapes may be used. The lead can have a solid or hollow body and may optionally include one or more lumens. In particular embodiments, a lead can be a relatively long implantable lead having a physical length of greater than about 10 cm (up to, for example, 1 m, or even longer). The lead can be an intrabody medical lead for acute or chronic use, including, for example, implantable leads. The lead can be for veterinary or human use.

The term “conductor” and derivatives thereof refer to a conductive trace, filar, wire, cable, flex circuit or other electrically conductive member. A conductor may also be configured as a closely spaced bundle of filars or wires. The conductor can be a single continuous length. The conductor can be formed with one or more of discrete filars, wires, cables, flex circuits, bifilars, quadrafilars or other filar or trace configuration, or by plating, etching, deposition, or other fabrication methods for forming conductive electrical paths. The conductor can be insulated. The conductor can also comprise any suitable MRI-compatible (and biocompatible) material such as, for example, MP35N drawn filled tubing with a silver core and an ETFE insulation on the drawn tubing.

The term “current suppression module” (“CSM”) refers to an elongate conductor that turns back on itself at least twice in a lengthwise direction to form a conductor configuration of a reverse or backward section in one lengthwise direction and proximately located forward sections that extend in the opposing lengthwise direction. The CSM can be configured with a length that is a sub-length of the overall length of the conductor, e.g., less than a minor portion of the length of the conductor, and the conductor can have multiple CSMs along its length. The term “MCSM” refers to a conductor that has multiple CSMs, typically arranged at different locations along at least some, typically substantially all, of its length. The terms “backward”, “rearward” and “reverse” and derivatives thereof are used interchangeably herein to refer to a lengthwise or longitudinal direction that is substantially opposite a forward lengthwise or longitudinal direction. The words “sections”, “portions” and “segments” and derivatives thereof are also used interchangeably herein and refer to discrete sub-portions of a conductor or lead.

The term “MRI compatible” means that the material is selected so as to be non-ferromagnetic and to not cause MRI operational incompatibility, and may also be selected so as not to cause undue artifacts in MRI images. The term “RF safe” means that the device, lead or probe is configured to operate within accepted heat-related safety limits when exposed to normal RF signals associated with target (RF) frequencies such as those frequencies associated with conventional MRI systems or scanners.

The term “high impedance” means an impedance that is sufficiently high to reduce, inhibit, block and/or eliminate flow of RF-induced current at a target frequency range(s). The impedance has an associated resistance and reactance as is well known to those of skill in the art. Some embodiments of the lead and/or conductors of the instant invention may provide an impedance of at least about 100 Ohms, typically between about 400 Ohms to about 600 Ohms, such as between about 450 Ohms to about 500 Ohms, while other embodiments provide an impedance of between about 500 Ohms to about 1000 Ohms or higher.

Embodiments of the invention configure leads that are safe (heat-resistant) at frequencies associated with a plurality of different conventional and future magnetic field strengths of MRI systems, such as at least two of 0.7 T, 1.0 T, 1.5 T, 2 T, 3 T, 7 T, 9 T, and the like, and that allow for safe use in those environments (future and reverse standard MRI Scanner system compatibility).

The term “tuned”, with respect to a coil, means tuned to define a desired minimal impedance at a certain frequency band(s) such as those associated with one or more high-field MRI Scanner systems. When used with respect to a parallel resonant circuit with inductive and capacitive characteristics defined by certain components and configurations, the word “tuned” means that the circuit has a high impedance at one or more target frequencies or frequency bands, typically including one or more MRI operating frequencies.

The term “coiled segment” refers to a conductor (e.g., trace, wire or filar) that has a coiled configuration. The coil may have revolutions that have a substantially constant diameter or a varying diameter or combinations thereof. The term “co-wound segments” means that the affected conductors can be substantially concentrically coiled at the same or different radii, e.g., at the same layer or one above the other. The term “co-wound” is used to describe structure indicating that more than one conductor resides closely spaced in the lead and is not limiting to how the structure is formed (i.e., the coiled segments are not required to be wound concurrently or together, but may be so formed).

The term “revolutions” refers to the course of a conductor as it rotates about its longitudinal/lengthwise extending center axis. A conductor, where coiled, can have revolutions that have a substantially constant or a varying (radius) distance from its center axis or combinations of constant and varying distances for revolutions thereof.

The term “Specific Absorption Rate” (SAR) is a measure of the rate at which RF energy is absorbed by the body when exposed to radio-frequency electromagnetic fields. The SAR is a function of input power associated with a particular RF input source and the object exposed to it, and is typically measured in units of Watts per kilogram (W/kg) taken over volumes of 1 gram of tissue or averaged over ten grams of tissue or over the entire sample volume, or over the volume of the exposed portion of the sample. SAR can be expressed as a peak input and/or whole body average value. Different MRI Scanners may measure peak SAR in different ways, resulting in some variation as is well known to those of skill in the art, while whole body average values are typically more consistent between different MR Scanner manufacturers.

Peak input SAR measurement is an estimate of the maximum input RF energy deposited in tissue during an MRI scan. To measure peak SAR, the following methodology using a suitable phantom can be employed. The peak SAR temperature(s) is typically measured near the surface. The phantom can be any shape, size and/or volume and is typically substantially filled with a medium simulating tissue, e.g., the medium has electrical conductivity corresponding to that of tissue—typically between about 0.1-1.0 siemens/meter. The medium can be a gel, slurry, or the like, as is well known, and has conduction and/or convective heat transfer mechanisms. Peak input SAR is estimated based on temperature rise measured by the sensors placed near the surface/sides of the phantom and is calculated by Equation 1 as stated below. See also, ASTM standard F2182-02A, which described a way to measure input SAR.

dT/dt=SAR/Cₚ Equation (1)

where:
dT is the temperature rise
dt is the change in time
Cₚ is the constant pressure specific heat of water (approx. 4180 J/kg-° C.).

The term “low DC resistance” refers to leads having less than about 1 Ohm, typically less than about 0.7 Ohm/cm, so, for example, a 60-70 cm lead can have DC resistance that is less than 50 Ohms. In some embodiments, a lead that is 73 cm long can have a low DC resistance of about 49 Ohms. Low DC resistance can be particularly appropriate for leads that connect power sources to certain components, e.g., electrodes and IPGs for promoting low-power usage and/or longer battery life.

The lead can have good flexibility and high fatigue resistance to allow for chronic implantation. For example, with respect to flexibility, the lead can easily bend over itself. In some embodiments, the lead, when held suspended in a medial location, is sufficiently flexible so that the opposing long segments drape or droop down together (do not hold a specific configuration).

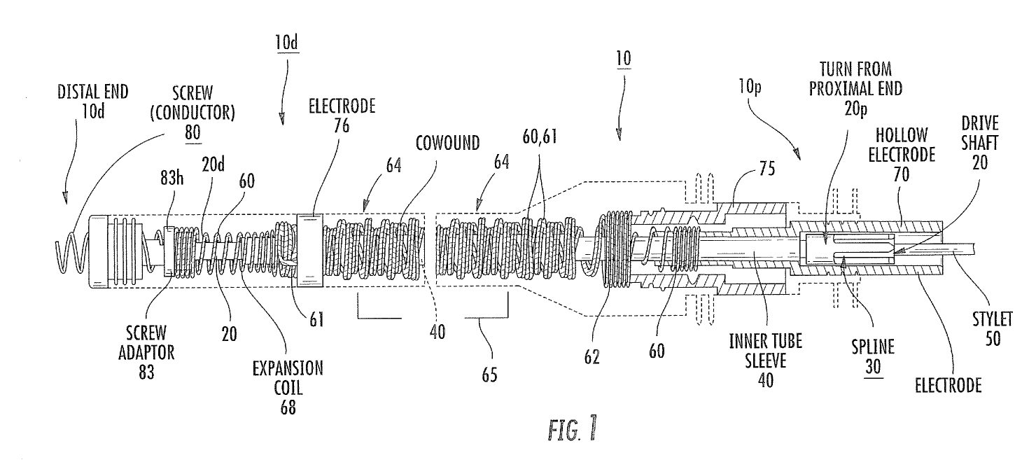

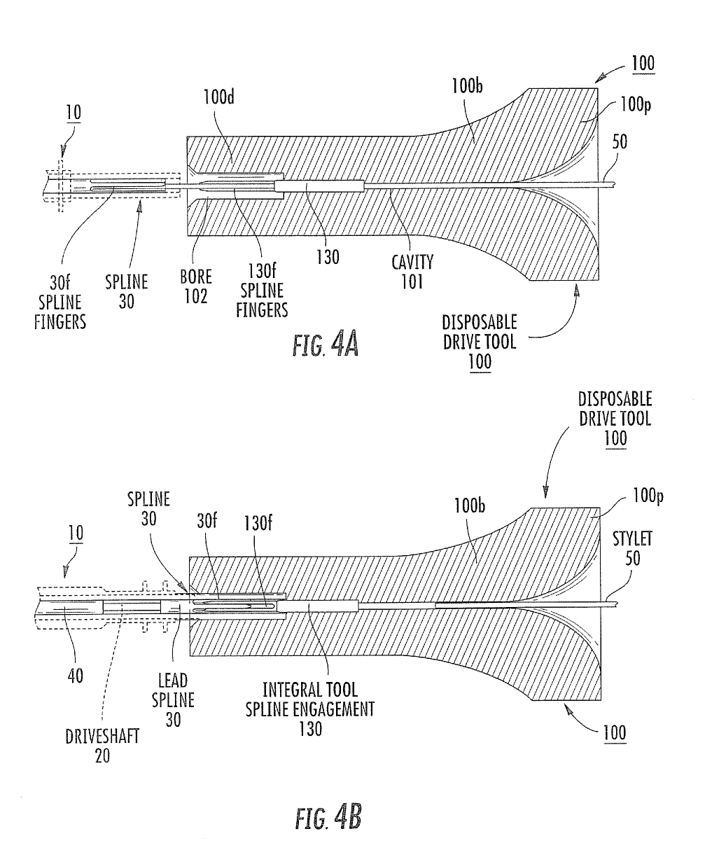

Turning now to the figures, FIGS. 1-3 and 5-8 illustrate an exemplary lead 10 with opposing proximal and distal end portions 10p, 10d, respectively. The lead 10 has an internal drive shaft 20 extending from the proximal end portion 10p to a distal end portion 10d. The proximal end portion of the drive shaft 20p can comprise a spline 30 or a spline engagement member that engages a spline of another releasably engageable shaft associated with a tool 100 (FIGS. 4A, 4B) used to position the lead in the body (e.g., for acute interventional therapy or chronic implantation). The tool 100 can include an integral spline or spline engagement member 130 (FIGS. 4A, 4B) that may cooperate with a stylet 50. The spline shaft of the tool when engaged to the lead drive shaft 20 is used to deploy the extendable member. For example, a clinician can linearly translate then rotate the tool 100 thereby rotating the member 130, which, in turn causes the drive shaft 20 of the lead to turn. The stylet 50 is optional but can provide additional rigidity to the lead during placement in the body. The distal end portion of the drive shaft 20d is in communication with a deployable or extendable member 80 that can be advanced and, optionally, retracted, in response to translation and rotation of the drive shaft 20. That is, clockwise or counterclockwise rotation of the drive shaft 20 can cause the target member 80 to rotate and advance out of the tip end of the lead (and, in some embodiments, rotation in the reverse direction can cause it to retract back into the tip or end of the lead).

As shown, the target extendable member is a screw 80. The screw can comprise a conductor material and the screw 80 can be attached to a screw adaptor 83 with a hub 83h. The screw 80 can also act as an electrode to transmit energy to local tissue. Rotation of the drive shaft 20 causes the screw conductor 80 to rotate and linearly translate between about 1 mm to about 1 cm. The tip nut 90 has internal threads that mesh with the screw, causing the screw to extend or retract when it rotates, along with the driveshaft and spline. The term “screw” refers to a member having a pointed substantially rigid spiral or helical fixation screw such as a corkscrew-like configuration as shown in the exemplary extendable member. The expansion coil 68 may connect a lead to the screw, while allowing for the translation and rotation of the screw by winding up or unwinding during the process. Although shown as a screw 80, the target extendable member 80 can be other members with other configurations, such as, for example, a needle, a sensor, a barb or anchor, a delivery device (drug or other therapy), a biopsy device, and the like.

The lead 10 can be a low profile lead with at least a major portion of its body having a cross-sectional area or diameter of about 0.20 inches or less. In some embodiments, the lead 10 is a low profile lead with a cross-sectional area or diameter that is between about 0.001 inches to about 0.085 inches over at least a portion of its length, e.g., such as at least a distal end portion of the lead 10d. In particular embodiments, the lead 10 can have a diameter or cross-sectional width or length that is between about 0.01 inches to about 0.18 inches over at least a major portion of its length, such as about 0.10 inches.

FIGS. 1-3 also illustrate that the lead 10 can have at least one electrode, shown as having three axially spaced apart electrodes, 70, 75, 76. At least one conductor extends to each of the electrodes 75, 76. The first electrode 70 can be a hollow electrode with a cavity that is sized and configured to receive the drive shaft 20 and spline 30. The second electrode 75 can extend over the hollow electrode 70. Each of the first and second electrodes 70, 75 can be fixed (e.g., static and non-rotating). The second electrode 75 can be affixed to the first electrode body 70 via adhesive or overmolding or the like. The space 71 between the two electrodes 70, 75 can be insulated, such as with silicone during a molding or overmolding process. The first electrode 70 can be affixed to the sleeve 40, which also is fixed (e.g., static and non-rotating). The drive shaft 20 and its proximal end 20p move or translate with respect to the sleeve and electrodes 70, 75.

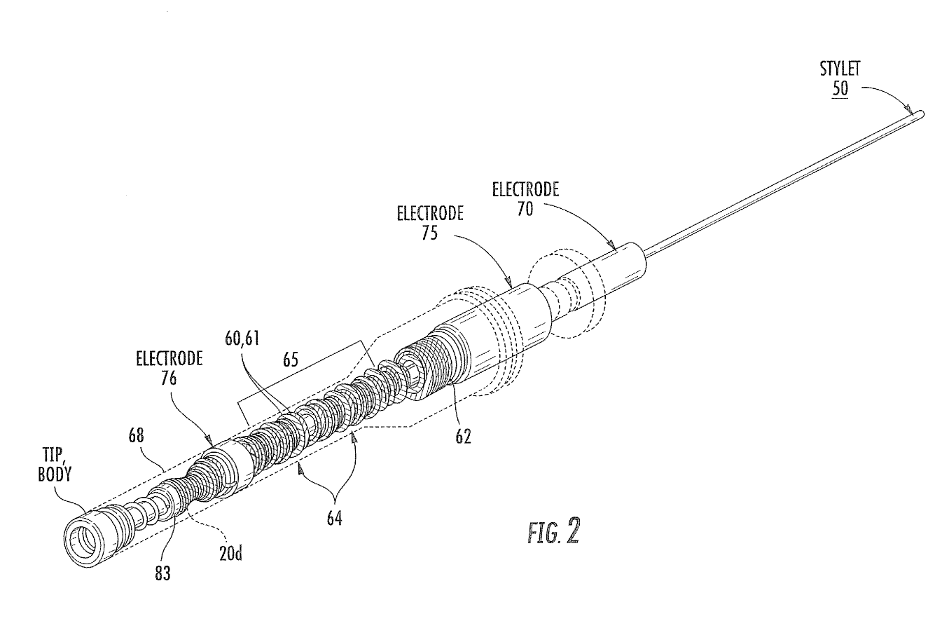

The expansion coil 68 can be defined by an extension or continuation of one or more of the conductors, shown as the inner conductor 60. Each conductor can include at least one CSM 64, shown as MCSMs 65 along their length as shown in FIGS. 1 and 2. The lead 10 can include two inner conductors 60, 61 that are cowound and define stacked (multi-layer coils) which are substantially concentric and turn lengthwise directions at least twice to form the CSMs 64. One of the conductors 60 can extend beyond the electrode 76 to form the expansion coil 68 and electrically connect the screw adaptor 83. The other conductor 61 terminates proximate the electrode 76.

The two inner conductors 60, 61 can reside over an inner flexible sleeve 40 as also shown in FIGS. 1-3. The inner conductors 60, 61 can be substantially concentric. The sleeve 40 can be static and be sized and configured to receive the drive shalt 20. The sleeve 40 can terminate in advance of the screw adaptor 83. For a discussion of fabrication methods and two and three-layer coil stacked coil configurations of one or more conductors, see, co-pending U.S. Patent Application Ser. No. 60/955,724, the contents of which are hereby incorporated by reference as if recited in full herein.

FIGS. 4A and 4B illustrate that the lead 10 can be slidably advanced into a distal end 100d of the tool body 100b. The tool body 100b can have a through cavity 101 which merges into a larger bore 102 containing fixed spline 130, that snugly and slidably receives and releasably engages the spline or spline engagement member 30 of the lead 10. The bore 102 can terminate into a stop position for secure engagement. The bore may have a countersunk lead-in edge to facilitate self-alignment. The drive tool 100 can be single-use disposable. A medical kit can be provided with a spare drive tool 100 in a sterile package for future use or the drive tool can be provided as a separate component so that a clinician can readily access the drive tool for future adjustment of the lead as appropriate (not shown). The tool body 100b can be ergonomically configured to allow a clinician to hold as a hand, finger or thumb tool for precisely advancing the extendable member. The tool body 100b and the lead can be MRI compatible and indeed, the tool body can be used to implant lead during an MRI interventional procedure.

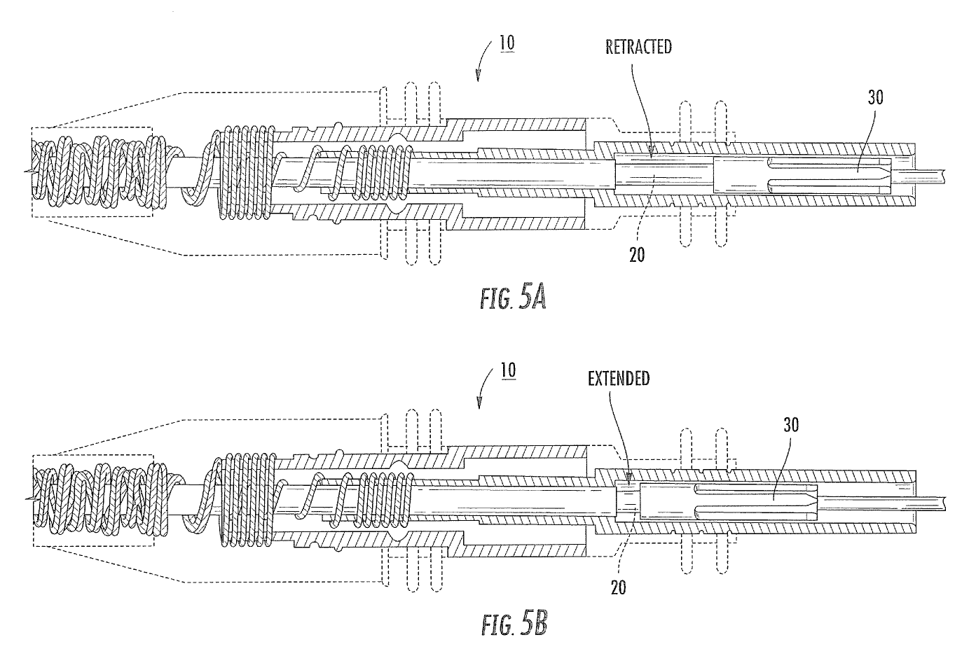

FIGS. 5A and 5B illustrate exemplary retracted and extended configurations of the drive shaft 30 in the lead 10, respectively. As shown in FIG. 5B, the drive shaft 20 can have a linear stroke distance “L” of suitable distance, such as, for example, between about 0.1 mm to about 1 cm.

FIGS. 4A and 4B also illustrate that both the lead 10 and tool 100 can include respective splines 30, 130 with each including a series of forwardly projecting fingers 30f, 10f that slide together to matably engage and allow the drive shaft to rotate while extending or retracting.

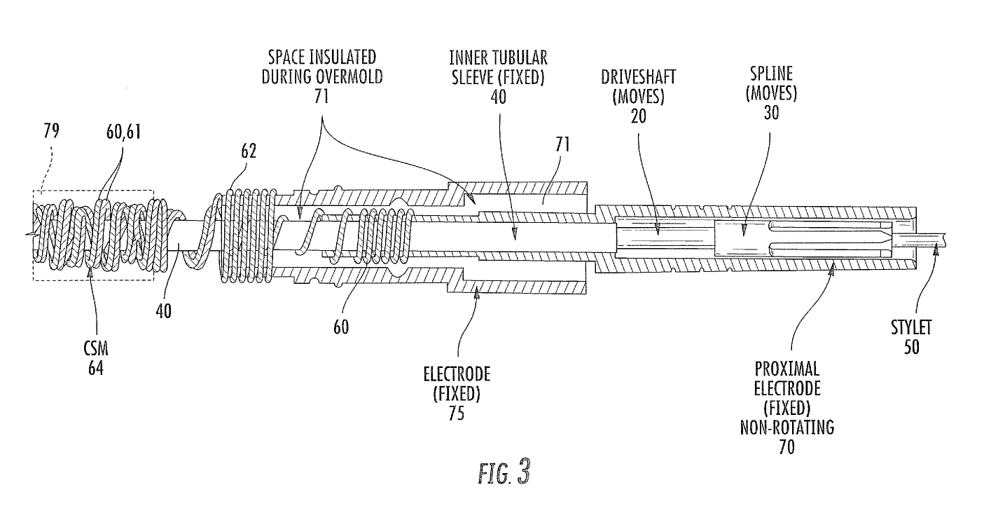

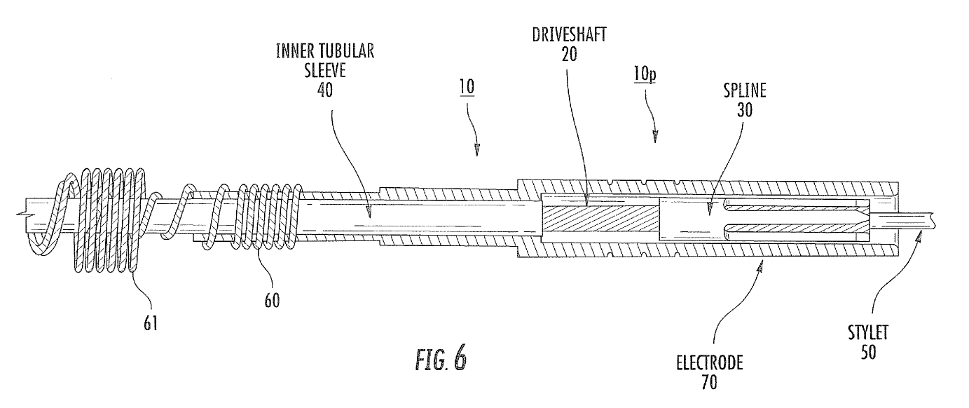

FIGS. 3 and 6 illustrates that the drive shaft 20 can comprise a substantially rigid polymer such as, for example, polyimide. The drive shaft 20 can have an inner diameter of less than about 0.028 inches, such as about 0.018 inches and an outer diameter of less than about 0.024 inches, such as about 0.021 inches. The spline 30 can comprise a substantially rigid material such as, for example, PEEK. The spline can have an inner diameter of about 0.022 inches and an outer diameter of about 0.035 inches. The stylet 50 can have a diameter that is about 0.014 inches. The inner sleeve 40 can be flexible and comprise a polymer material such as for example, nylon, HDPE or FEP and can have an inner diameter of about 0.024 inches and an outer diameter of about 0.028 inches. The electrode 70 can have an outer diameter of about 0.063 inches and an inner diameter that slidably receives the spline. The electrode 75 can have an outer diameter of about 0.105 inches and the electrode 76 can have an outer diameter of about 0.084 inches. The distal end of the lead 10d can have a diameter of about 0.084 inches (with a substantially constant outer diameter from at least about the electrode 76, and typically from beyond electrode 75 to the tip). Other configurations/sizes and materials for the lead, shaft, spline, sleeve, electrode(s) and stylet may be used. In some embodiments, the stylet is not required. The stylet 50 or another elongate member can be used to facilitate alignment/lateral centering of the two shafts for ease of engagement.

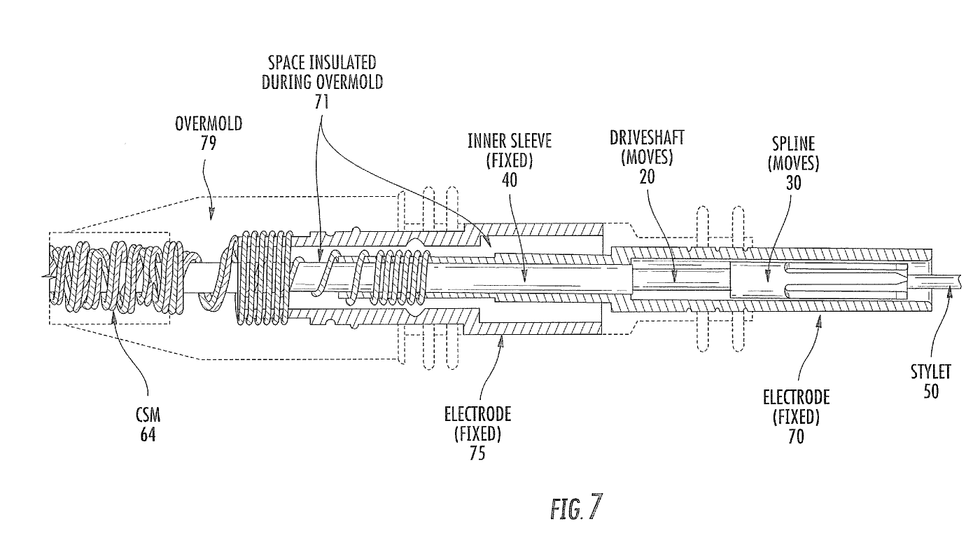

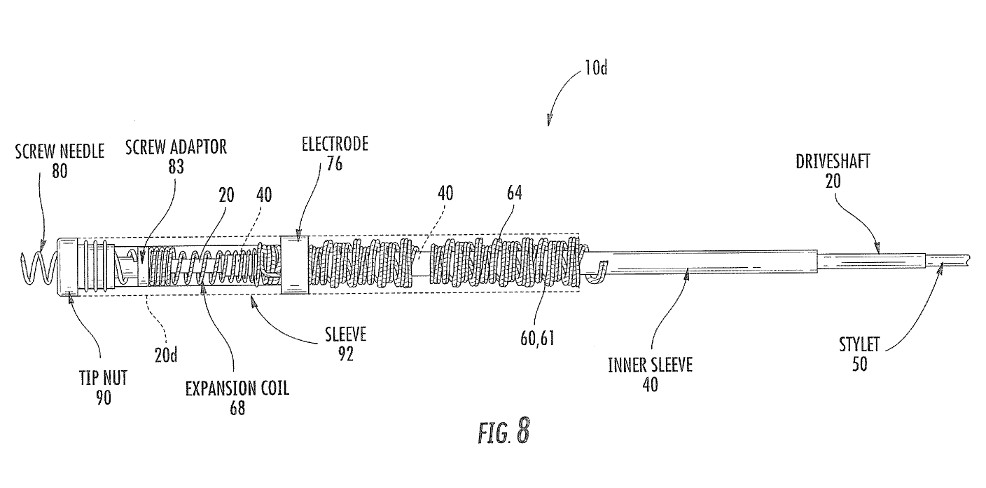

FIG. 7 illustrates that the distal end of the lead 10 can have a tip nut 90 with internal threads that mesh with the screw and that as shown in FIG. 8, the expansion coil can reside in a silicone or other biocompatible sleeve 92, which allows the expansion coil to wind up or unwind during extension or retraction of the screw. Similarly, the lead 10 can be encased in a biocompatible material such as silicone overmold 79 as shown in FIG. 7 to have the desired profile shape or size.

In some embodiments, the lead 10 can be a neuromodulation lead or a cardiac lead. The lead can be an implantable lead such as a pacemaker lead. Embodiments of the invention can be particularly suitable for an active fixation bradyarrhythmia lead. The lead can include a distal electrode conductor 61 and/or 62 wound in a two-layer or trilayer CSM 64 along the length of the lead. The proximal electrode conductor 62 can be substantially concentrically arranged outside the distal electrode conductors 60, 61.

Although the above has primarily described the drive shaft in connection with a lead, the invention is not limited thereto and may be use with any medical device desiring a drive tool. For example, the features of the invention can be used with a catheter, probe or the like.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. The invention is defined by the following claims, with equivalents of the claims to be included therein.

That which is claimed:

1. A medical device comprising:
an elongate body having opposing proximal and distal end portions with an axially extending center cavity;
an internal drive shaft residing in the center cavity, the drive shaft having a proximal end portion with a rotatable spline residing in the proximal end portion of the elongate body; and
an extendable member held in the distal end portion of the elongate body, the extendable member in communication with the drive shaft, whereby rotation of the drive shaft causes the extendable member to extend out of the elongate body.

2. A medical device according to claim 1, wherein the elongate body is an intrabody medical lead with at least one conductor, and wherein the lead has low DC resistance and is flexible.

3. A medical device according to claim 1, wherein the extendable member comprises a screw electrode.

4. A medical device according to claim 1, wherein the extendable member is an electrode or sensor, wherein the medical device comprises at least one conductor in electrical communication with the electrode or sensor, and wherein the elongate body is an implantable lead and has a diameter that is less than about 0.10 inches over at least a major portion of its length.

5. A medical device according to claim 1, wherein the elongate body is an implantable neuromodulation lead.

6. A medical device according to claim 1, wherein the elongate body is an implantable cardiac lead.

7. A medical device according to claim 1, wherein the elongate body is an implantable pacemaker lead.

8. A medical device according to claim 1, wherein the elongate body comprises a flexible inner sleeve residing over the drive shaft and at least one conductor portion coiled substantially concentrically about the sleeve.

9. A medical device according to claim 1, wherein the elongate body comprises a stationary electrode disposed on the proximal end portion of the elongate body about the drive shaft spline.

10. A medical device according to claim 1, in combination with a single-use disposable drive tool, the drive tool having a primary body with an axially extending cavity and a spline and/or spline engagement member residing in a distal end portion of the drive tool primary body, the tool spline and/or spline engagement member of the drive tool adapted to slidably releasably engage the elongate body drive shaft spline whereby a user can rotate the drive shaft.

11. A medical device and tool according to claim 10, wherein the drive tool primary body has a bore sized and configured to snugly slidably receive the proximal end portion of the elongate body, wherein the bore has a larger diameter than the axially extending cavity.

12. A medical device and tool according to claim 10, further comprising a stylet that extends out of the proximal end portion of the drive tool and resides in a center cavity extending through the drive shaft, wherein a user rotates the tool to rotate and translate the drive shaft.

13. A surgical tool set, comprising:
a flexible intrabody medical lead, probe or catheter having an internal drive shaft with a spline or spline engagement member, the lead, probe or catheter also comprising a plurality of electrodes, and a plurality of conductors, each electrode in communication with at least one of the conductors; and
a drive tool having an internal spline or spline engagement member sized and configured to slidably releasably engage the internal drive shaft spline or spline engagement member, the drive tool configured to allow a user to rotate the drive shaft of the medical lead, probe or catheter.

14. A tool set according to claim 13, wherein the flexible lead and the drive tool each include a center cavity that slidably snugly receive a stylet.

15. A tool set according to claim 13, further comprising a second drive tool held in a discrete sterile package for future use.

16. A tool set according to claim 13, wherein the lead, probe or catheter is an implantable pacemaker lead.

17. A tool set according to claim 16, wherein the lead, probe or catheter is a lead that comprises an active fixation device on a distal end thereof in communication with the drive shaft configured to attach to local tissue, wherein, in use, the drive tool spline rotates the drive shaft causing the active fixation device to rotate and extend out of the lead.

18. A method of advancing an extendable member from a medical lead, comprising:
matably engaging an integral spline of a disposable single-use drive tool with an intrabody medical lead having an internal drive shaft and spline;
turning the drive tool to rotate the drive shaft; and
rotating the extendable member from the lead in response to the turning step.

19. A method according to claim 18, further comprising turning the tool in a direction opposite of that used to advance the extendable member and retracting the extendable member back into the lead in response thereto.

20. An implantable pacemaker lead comprising:
a pacemaker lead having opposing proximal and distal end portions with an axially extending center cavity;
an internal rotatable and translatable drive shaft residing in the center cavity, the drive shaft having a proximal end portion with a rotatable spline, the spline residing in the proximal end portion of the lead; and
an extendable member held in a retracted configuration in the distal end portion of the lead, the extendable member in communication with the drive shaft, whereby rotation of the drive shaft causes the extendable member to extend out of the lead.

21. A pacemaker lead according to claim 20, wherein the lead has low DC resistance and is flexible, and wherein the extendable member comprises a fixation screw, and wherein the lead is an active fixation bradyarrhythmia lead with a distal electrode.

22. A single-use disposable medical drive tool having an internal spline or spline engagement member sized and configured to slidably releasably receive and engage an end portion of an intrabody medical lead having an internal drive shaft with an end having a spline or spline engagement portion configured to engage the drive tool spline or spline engagement member, the drive tool further comprising a cavity for receiving a stylet, the tool being configured to allow a user to both rotate and translate the internal drive shaft of the medical lead, wherein the medical drive tool is held in a sterile package.
